(19) United States
(12) Patent Application Publication
(10) Pub. No.: US 2023/0013336 A1
(43) Pub. Date: Jan. 19, 2023

(54) SYNECHOCOCCUS ELONGATUS MUTANTS, VARIANTS AND USES THEREOF TO PRODUCE AN ESSENTIAL AMINO ACID

(71) Applicant: Purdue Research Foundation, West Lafayette, IN (US)

(72) Inventors: John Morgan, West Lafayette, IN (US); Arnav Deshpande, Broomfield, CO (US)

(21) Appl. No.: 17/859,837

(22) Filed: Jul. 7, 2022

Related U.S. Application Data
(60) Provisional application No. 63/219,691, filed on Jul. 8, 2021.

(51) Int. Cl. C12P 13/22; C12N 1/20; C12N 15/01

(52) U.S. Cl. CPC C12P 13/222; C12N 1/205; C12N 15/01

(57) ABSTRACT

A method of generating a variant cyanobacterium (e.g., Synechococcus elongatus, in particular S. elongatus sp. PCC11801) for photoautotrophic production of an amino acid (e.g., L-phenylalanine); the variant so produced; a method of extending growth of a culture of a variant cyanobacterium and its photoautotrophic production of an amino acid; and a method of photo-autotrophically producing L-phenylalanine.

### PRIORITY

The present application is related to and claims the priority benefit of U.S. Provisional Patent Application No. 63/219,691 filed Jul. 8, 2021, the content of which is hereby expressly incorporated by reference in its entirety into this disclosure.

### TECHNICAL FIELD

This disclosure generally relates to cyanobacteria, specifically Synechococcus elongatus, mutagenesis, S. elongatus mutants, variants, and cultures thereof, and photoautotrophic amino acid production.

### BACKGROUND

Cyanobacteria, a group of photosynthetic prokaryotes, are attractive hosts for biotechnological applications, at least in part because cyanobacteria can convert carbon dioxide (CO₂) to biochemicals and biofuels directly via light-driven, endergonic reactions. However, due primarily to slow growth, initial engineering efforts have resulted in low productivity and titers compared to heterotrophs. Indeed, while engineered cyanobacteria have been reported for the production of a wide variety of chemicals, such production is at titers that are well below those needed for commercial production. The majority of these studies employed model strains such as Synechocystis sp. PCC 6803, Synechococcus elongatus PCC 7942, and Synechococcus sp. PCC 7002. Substantial information is publicly available on transcriptome, proteome, metabolome, and synthetic biology tools of these model cyanobacteria.

Recently, a robust, fast-growing strain of cyanobacteria that is resistant to abiotic stresses was reported (Jaiswal et al. (2018), infra). Under ideal conditions Synechococcus elongatus sp. PCC 11801 grows at a rate comparable to Baker's yeast (e.g., with a doubling time of 2.3 hours under ambient CO₂ conditions and even faster under higher CO₂ levels and temperatures).

If Synechococcus elongatus, in particular S. elongatus sp. PCC 11801, could be modified to overproduce a biochemical, such as an amino acid (e.g., an essential amino acid), it would advance the utilization of cyanobacteria as microbial factories.

In view of the above, it is an object of the present disclosure to provide methods for producing S. elongatus mutants, in particular mutants of S. elongatus sp. PCC 11801, for production of a biochemical, such as an amino acid (e.g., an essential amino acid, such as L-phenylalanine). Moreover, in certain cases, it would be beneficial to provide such a mutant that is tolerant to high temperatures, light, and CO₂ levels, considering potential outdoor utilization in biorefineries. These and other objects and advantages will be apparent from the detailed description provided herein.

### SUMMARY

A method of generating a variant cyanobacterium for photoautotrophic production of an amino acid is provided. In certain embodiments, the method comprises inducing mutagenesis in a wild-type Synechococcus elongatus sp. strain by exposing the wild-type Synechococcus elongatus sp. strain to methylmethanesulfonate (MMS) or ultraviolet (UV) irradiation, or both MMS and UV irradiation to generate a mutant Synechococcus elongatus sp. strain; and contacting the mutant Synechococcus elongatus sp. strain with an amino acid analog (e.g., a phenylalanine analog) and selecting a variant of the mutant Synechococcus elongatus sp. strain having increased production of the amino acid and less than about 5% reduction in biomass accumulation as compared with the wild-type Synechococcus elongatus sp. strain.

In certain embodiments, the method comprises inducing mutagenesis in a wild-type Synechococcus elongatus sp. strain by exposing the wild-type Synechococcus elongatus sp. strain to MMS, UV irradiation, or both MMS and UV irradiation to generate a mutant Synechococcus elongatus sp. strain; and contacting the mutant Synechococcus elongatus sp. strain with an amino acid analog and selecting a variant of the mutant Synechococcus elongatus sp. strain having increased production of the amino acid and less than about a 5% reduction in biomass accumulation as compared with the wild-type Synechococcus elongatus sp. strain.

The wild-type Synechococcus elongatus can be Synechococcus elongatus sp. PCC11801, for example. The amino acid can be phenylalanine. The amino acid can be L-phenylalanine. The amino acid can be tryptophan. In certain embodiments where the amino acid is phenylalanine or L-phenylalanine, the amino acid analog is 3-(2-thienyl)-DL-alanine.

Contacting the mutant strain with an amino acid analog and selecting a variant of the mutant Synechococcus elongatus sp. strain can, in certain embodiments, further comprise: plating the mutant Synechococcus elongatus sp. strain on agar containing the amino acid analog and selecting a first variant colony of the mutant Synechococcus elongatus sp. strain that grows in the presence of the amino acid analog; replating the selected first variant colony and selecting at least a second variant colony of the mutant Synechococcus elongatus sp. strain that grows in the presence of the amino acid analog to obtain segregated variant colonies; transferring the segregated variant colonies to a liquid medium containing the amino acid analog and selecting for segregated variants that grow in the presence of increasing concentrations of the amino acid analog; sub-culturing variants from the selected segregated variants in the liquid medium containing the amino acid analog for at least about 24-72 hours; and selecting a sub-cultured variant for increased production of the amino acid and less than 5% reduction in biomass accumulation as compared with the wild-type Synechococcus elongatus sp. strain. The liquid medium can be, for example, BG-11 medium. In certain embodiments, the BG-11 medium is modified to contain increased concentrations of magnesium sulfate heptahydrate, sodium nitrate, potassium phosphate dibasic, and A5 mineral solution and to include ammonium chloride (BG-11M medium).

Additionally, the method can further comprise: inducing mutagenesis in the selected sub-cultured variant by exposing the selected sub-cultured variant to MMS, UV irradiation, or both MMS and UV irradiation to generate a sub-cultured mutant; and contacting the selected sub-cultured mutant with an amino acid analog and selecting a variant of the selected sub-cultured mutant having increased production of the amino acid and less than about a 5% reduction in biomass accumulation as compared with the wild-type Synechococcus elongatus sp. strain. For example, the selected sub-cultured mutant can be exposed to MMS and UV irradiation for a period of about 60 seconds to about 120 seconds.

In certain embodiments, the method can further comprise incubating the mutant Synechococcus elongatus sp. strain for at least about 8 hours under dark, heated conditions (e.g., wherein heated conditions are about 38° C. to about 40° C.).

Contacting the mutant strain with an amino acid analog can be performed in the presence of light and air supplemented with about 3% v/v carbon dioxide (CO₂).

The method comprises:

(a) exposing a culture of wild-type Synechococcus elongatus to MMS and/or UV irradiation to generate a mutagenized culture;

(b) incubating the mutagenized culture for at least 8 hours under dark, heated conditions;

(c) plating the mutagenized culture on agar containing an amino acid analog and selecting mutant colonies that grow in the presence of the amino acid analog;

(d) replating the mutant colonies selected in (c) and selecting mutant colonies that grow in the presence of the amino acid analog;

(e) replating the mutant colonies selected in (d) and selecting mutant colonies that grow in the presence of the amino acid analog; and, optionally, replating the mutant colonies selected in (e) and selecting mutant colonies that grow in the presence of the amino acid analog, to obtain genetically segregated mutant colonies;

(f) transferring individual mutant colonies to a liquid medium containing the amino acid analog and selecting segregated mutants that grow in the presence of increasing concentrations of the amino acid analog;

(g) sub-culturing mutants from (f) in the liquid medium containing the amino acid analog for at least about 24-72 hours;

(h) quantifying the amino acid titer in a supernatant obtained from a sub-cultured mutant; and

(i) selecting a sub-cultured mutant for increased production of the amino acid and less than 5% reduction in biomass accumulation. The method can further comprise:

(j) obtaining a mutant of the sub-cultured mutant selected in (i) that outperforms the sub-cultured mutant selected in (i) for production of the amino acid. The wild-type Synechococcus elongatus can be Synechococcus elongatus sp. PCC11801. The amino acid can be L-phenylalanine, in which case the amino acid analog can be 3-(2-thienyl)-DL-alanine; aromatic amino acids can be quantified in (h). The liquid medium can be BG-11 medium. The BG-11 medium can be BG-11M medium (i.e., modified to contain increased concentrations of magnesium sulfate heptahydrate, sodium nitrate, potassium phosphate dibasic, and A5 mineral solution and to include ammonium chloride). The incubated conditions in (b) can be about 30° C. to about 40° C.

Also provided is a variant of Synechococcus elongatus obtained by the above methods, such as when the culture in (a) was exposed to UV irradiation, MMS, or both UV irradiation and MMS. The variant Synechococcus elongatus can be a mutagenized Synechococcus elongatus sp. PCC11801. The variant can produce at least about 0.5 g/L of L-phenylalanine after three days of culture, such as with a biomass production of at least about 95% that of the wild-type strain (such as at least about 95% of the wild-type strain), e.g., when cultured in BG-11M medium.

When the method further comprises (j), the variant can produce at least about 0.8 g/L, at least about 1.2 g/L, or up to about 2.0 g/L of L-phenylalanine after three days of culture, such as with a biomass production of at least about 95% that of the wild-type strain, e.g., when cultured in BG-11M medium. The variant can produce about 3.0 g/L (such as 3.0 g/L) of L-phenylalanine after about 15 days of culture, such as with a biomass production of at least about 95% that of the wild-type strain, e.g., when cultured in BG-11 medium that is periodically replenished.

A method of extending growth of a culture of a mutant cyanobacterium and its photoautotrophic production of an amino acid is also provided. The method comprises:

(a) culturing the mutant cyanobacterium, which photo-autotrophically produces an amino acid, in BG-11 medium,

(b) supplying the culture with air supplemented with CO₂, and

(c) periodically replenishing the BG-11 medium. The BG-11 medium can be BG-11M medium. The exogenous CO₂ can be at least about 3% v/v (such as at least 3% v/v). The BG-11 medium can be replenished every 2-3 days, such as on days 3, 6, 9, 12, and 15. The variant cyanobacterium can be a mutagenized Synechococcus elongatus, such as one that is selected for overproduction of L-phenylalanine. The variant Synechococcus elongatus can be a mutant Synechococcus elongatus sp. PCC11801, such as one that is selected for overproduction of L-phenylalanine. The culture of a variant of S. elongatus sp. PCC11801 can accumulate at least about 7 g/L of biomass and at least about 3 g/L (such as 3.0 g/L) of L-phenylalanine at 15 days in culture. The culture can be viable for longer than 15 days, such as 20 days.

Further provided is a method of photo-autotrophically producing L-phenylalanine. The method comprises:

culturing a variant of S. elongatus sp. strain by exposing the wild-type Synechococcus elongatus sp. strain to MMS, UV irradiation, or both MMS and UV irradiation to produce a mutant Synechococcus elongatus sp. strain, and contacting the mutant Synechococcus elongatus sp. strain with an L-phenylalanine analog and selecting a variant of the mutant Synechococcus elongatus sp. strain having increased L-phenylalanine production and less than about a 5% reduction in biomass accumulation as compared with the wild-type Synechococcus elongatus sp. strain.

In certain embodiments, the selected variant can produce at least about 0.5 g/L, at least about 0.8 g/L, or at least about 2.0 g/L of L-phenylalanine after three days of culture or about 3.0 g/L (such as 3.0 g/L) of L-phenylalanine after 15 days of culture, in BG-11 or BG-11M medium in the presence of light and air supplemented with about 3% v/v (such as 3% v/v) CO₂. The BG-11 or BG-11M medium can be replenished every 2-3 days, such as on days 3, 6, 9, 12, and 15. The method can further comprise collecting phenylalanine (e.g., L-phenylalanine) from a biomass of the cultured variant or a culture medium thereof.

### DESCRIPTION OF FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1 shows a diagram of a simplified shikimate pathway, with the pathway beginning with condensation of erythrose-4-phosphate (E4P) (a pentose phosphate pathway (PPP) intermediate) and phosphoenolpyruvate (PEP) (a glycolytic intermediate), and where DAHP is 3-deoxy-D-arabinoheptulosonate 7-phosphate, SEEK is shikimate, CHA is chorismate, ANT is anthranilate, TRP is tryptophan, PPA is prephenate, PHE is phenylalanine, TYR is tyrosine, DAHPS is 3-deoxy-D-arabinoheptulosonate 7-phosphate synthase, AS is anthranilate synthase, CM is chorismate mutase, and PD is prephenate dehydratase.

FIG. 2 shows a diagram of a strain development strategy generally representative of the methods herein.

FIG. 3 shows phenylalanine accumulation in the supernatants from selected mutant cyanobacteria after three days of culture. The strains were grouped by the mutagen from which they were derived; ultraviolet (UV) irradiation (M11-M21) and methylmethanesulfonate (MMS) (M1-M10). Growth conditions were 250 mL Erlenmeyer shake flasks with 50 mL BG-11M medium starting at OD₇₃₀ 0.05 at 38° C., 240 μmol photons m⁻² s⁻¹ light intensity, 200 rpm and ambient carbon dioxide (CO₂). The concentrations of 3-(2-thienyl)-dl-alanine used as selection pressure for mutants are shown in the legend.

FIG. 4A shows a graph of PHE production of MMS and UV mutants without analog selection pressure after five days in culture at 240 μmol photons m⁻² s⁻¹ light intensity under ambient CO₂. Analog resistance in the graph indicates the original concentration on selection plates and does not indicate that the media contained analog. Error bars indicate the standard deviation from three biological replicates.

FIG. 4B shows the biomass accumulation of wild-type (WT), mutant variant M14, and mutant variant M14.2 at the end of three days of culture in 250 mL Erlenmeyer shake flasks with 50 mL BG-11M medium starting at OD₇₃₀ 0.2 at 38° C., 240 μmol photons m⁻² s⁻¹ light intensity, 200 rpm and 3% CO₂.

FIG. 4C shows the phenylalanine titer of WT, M14, and M14.2 at the end of three days of culture in 250 mL Erlenmeyer shake flasks with 50 mL BG-11M medium starting at OD₇₃₀ 0.2 at 38° C., 240 μmol photons m⁻² s⁻¹ light intensity, 200 rpm and 3% CO₂ (i.e. total capacity of biomass and PHE sinks) (* indicates p<0.05 using a two tailed two sample t-test).

FIG. 5A shows the PHE accumulation of overproducing M14.2 in 250 mL Erlenmeyer shake flasks with 50 mL BG-11M medium starting at OD₇₃₀ 0.2 at 38° C., 240 μmol photons m⁻² s⁻¹ light intensity, 200 rpm and 3% CO₂, with and without nutrient replenishment on days 3, 6, 9, 12 and 15 (addition of complete BG-11M nutrients using a concentrated solution to minimize volume change).

FIG. 5B shows the growth of M14.2 in 250 mL Erlenmeyer shake flasks with 50 mL BG-11M medium starting at OD₇₃₀ 0.2 at 38° C., 240 μmol photons m⁻² s⁻¹ light intensity, 200 rpm and 3% CO₂, with and without nutrient replenishment on days 3, 6, 9, 12 and 15 (addition of complete BG-11M nutrients using a concentrated solution to minimize volume change).

FIG. 6A shows a line graph of PHE production of WT, mutant variant M14, and mutant variant M14.2 under a 12-hour light (240 μmol photons m⁻² s⁻¹) and 12-hour dark cycle under ambient CO₂. Error bars indicate standard deviation of three biological replicates.

FIG. 6B shows a bar graph of both biomass and PHE production of WT, mutant variant M14, and mutant variant M14.2 under 12-hour light (240 μmol photons m⁻² s⁻¹) and 12-hour dark cycle under ambient CO₂ after 84 hours in culture. Error bars indicate standard deviation of three biological replicates.

FIG. 7A and FIG. 7B are comparisons of PHE productivity in engineered cyanobacteria (FIG. 7A) and total carbon sink productivities in cyanobacteria (FIG. 7B).

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles of the present disclosure, reference will now be made to the embodiments illustrated in the drawings and specific language will be used to describe the same. No limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the assemblies, compositions, and methods hereof can comprise many different configurations, forms, materials, and accessories.

Cyanobacteria can be useful in altering metabolic pathways or artificially regulating metabolites through genetic modification. Specifically cyanobacteria can fix carbon dioxide (CO₂) and convert it into valuable biochemicals. The present disclosure is predicated, at least in part, on the mutagenesis of a recently discovered, robust, fast-growing strain of cyanobacteria known as Synechococcus elongatus sp. PCC 11801.

Given the limited availability of synthetic biology tools, in the present disclosure, random mutagenesis (e.g., methylmethanesulfonate (MMS) or ultraviolet (UV) radiation) is coupled with amino acid analog selection (e.g., a phenylalanine analog) to produce a variant strain that overproduces a biochemical, such as an amino acid, such as an aromatic amino acid, in particular phenylalanine. As used herein, the term “strain” refers to any culture, generally pure culture, of a microorganism such as a cyanobacteria species (including a Synechococcus elongatus sp. strain) obtained from a single cell or an isolated colony.

Phenylalanine (Phe) is an aromatic amino acid synthesized by the shikimate pathway with applications in at least the food, feed, and pharmaceutical industries. In certain embodiments, the mutant strain can overproduce L-phenylalanine.

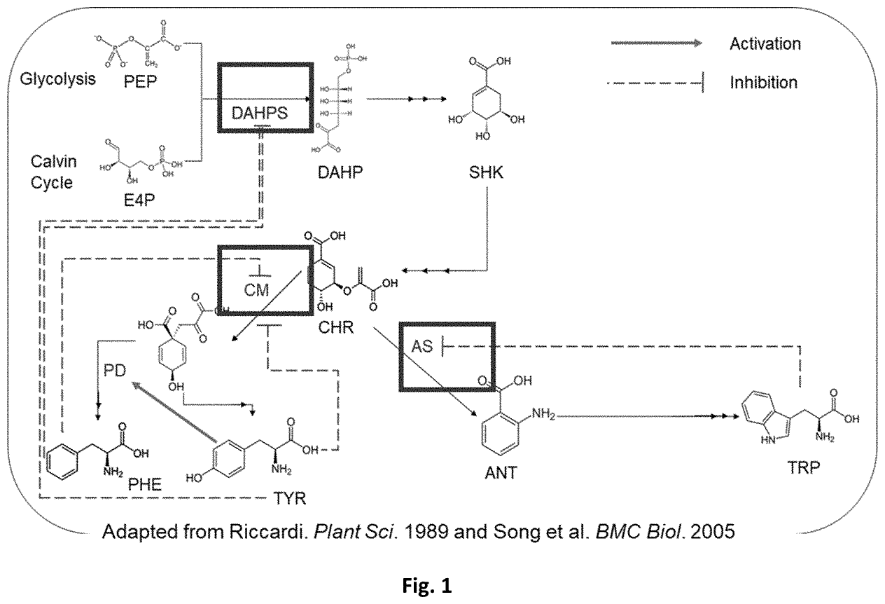

The flux towards amino acid biosynthesis in the shikimate pathway is tightly regulated in cyanobacteria by allosteric feedback regulation (Riccardi et al. (1989), infra). FIG. 1 shows a diagram of a simplified shikimate pathway, with the key rate limiting steps being catalyzed by 3-deoxy-D-arabino-heptulosonate-7-synthase (DAHPS) and prephenate dehydratase (PD). The present method bypasses and/or removes this regulation to relieve feedback inhibition and result in an upregulation of amino acid (e.g., phenylalanine) production. Although chorismite mutase (CM) has been shown to be regulated by phenylalanine and tyrosine in a few cyanobacterial strains, it is unlikely to be the case in PCC 11801 as CM is only 130 amino acids long and does not contain the phenylalanine binding site.

The production of biochemicals, such as amino acids, in particular L-phenylalanine, in cyanobacteria, such as Synechococcus elongatus, in particular Synechococcus elongatus sp. PCC11801, can obviate the need to use an organic carbon source derived from plants. This avoids taking land space away from growing crops that are used as animal (including human) food sources. Also, given that cyanobacteria can be grown in raceway ponds on non-arable land, there would be no competition for land space with crop plants used as food sources. Raceway ponds are much less expensive than bioreactors, which are used to culture E. coli. Light and CO₂ are freely available from the atmosphere, too, thereby allowing for “photo-autotrophic production.” Since cyanobacteria use a single-step process (i.e., direct conversion of CO₂ and light into an amino acid, such as an aromatic amino acid, e.g., L-phenylalanine), instead of a two-step process (i.e., obtention of glucose from a plant source and heterotrophic fermentation), the use of cyanobacteria to produce L-phenylalanine potentially allows for carbon-offset credits for production. The avoidance of chemical synthesis, such as the Strecker method, avoids the use of KCN and the reaction of aldehyde and ammonium chloride, which is undesirable and not cost-effective.

The aforementioned advantages are increasingly important in view of ongoing increases in production of biochemicals, such as amino acids. In 2017, 43% of production was for animal (e.g., cattle, pigs, and poultry) feed at a market value of $25-40/kg. Other areas where production requirements are increasing include aquaculture, precursors, drugs, nutraceuticals and artificial sweeteners. In addition, upon harvesting of the L-phenylalanine from the culture medium, the biomass can be used as an animal feed or aquaculture supplement. As used herein, “biomass” means a culture or cells in culture medium of cyanobacteria species, such as a Synechococcus elongatus sp. strain, a variant of a Synechococcus elongatus sp. strain, etc. as disclosed herein. Alternatively, the term “biomass” refers to at least partially dewatered algal or microalgal culture, or dewatered culture.

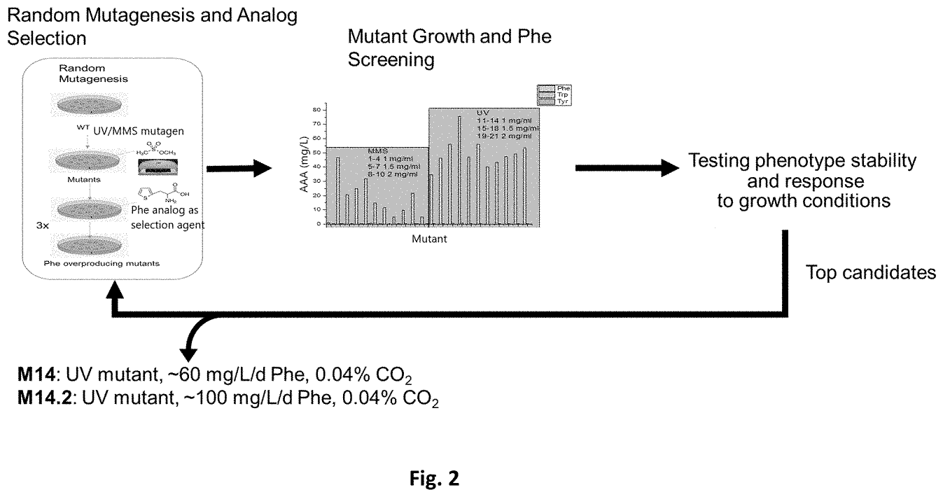

In view of the above, a method of generating a variant cyanobacterium for photoautotrophic production of an amino acid is provided. In certain embodiments, such methods can produce fast growing cyanobacterial strains for the sustainable production of valuable biochemicals using CO₂ and sunlight. As shown in FIG. 2, such methods can have three general categories of steps: random mutagenesis to generate a mutant strain and analog selection, mutant growth and amino acid screening to identify one or more mutant variants (i.e., each a “variant”) of interest, and testing the selected variants' phenotype stability and response to growth conditions. As used herein, the term “variant” or “mutant” of a reference strain X refers to any strain obtained from the reference strain X. The term “variant” more particularly refers to a strain obtained mainly by mutation and selection performed on a reference strain X, and the term “mutant” more particularly refers to a strain obtained by random mutagenesis (e.g., MMS and/or UV irradiation) applied to a reference strain X. Once a mutant or variant possesses the features according to various aspects disclosed herein, particularly a higher or increased amino acid (e.g., phenylalanine) content as compared with a naive cyanobacteria species, particularly a wild-type Synechococcus elongatus sp. strain, it falls within the protection scope claimed.

The method can comprise inducing mutagenesis in a wild-type Synechococcus elongatus sp. strain by exposing the wild-type Synechococcus elongatus sp. strain to MMS or UV irradiation, or both MMS and UV irradiation to generate a mutant Synechococcus elongatus sp. strain; and contacting the mutant Synechococcus elongatus sp. strain with an amino acid analog (e.g., a phenylalanine analog) and selecting a variant of the mutant Synechococcus elongatus sp. strain having increased production of the amino acid and less than 5% reduction in biomass accumulation as compared with the wild-type Synechococcus elongatus sp. strain.

Additionally, the method can further comprise inducing mutagenesis in the selected variant strain by exposing the selected variant strain to MMS or UV irradiation, or both MMS and UV irradiation to generate a secondary mutant. There, the method can further comprise contacting the secondary mutant with an amino acid analog and selecting a variant of the secondary mutant having increased production of the amino acid (e.g., as compared to the wild-type strain and/or the selected (first) variant strain). For example, the secondary mutant can be exposed to MMS and UV irradiation for a period of about 60 seconds to about 120 seconds, such as about 60 seconds to 120 seconds or 60 seconds to about 120 seconds. In certain embodiments, an additional selection criterion for the secondary mutant can also be less than a 5% reduction in the biomass accumulation as compared to the wild-type Synechococcus elongatus sp. strain.

In certain embodiments, contacting the mutant strain with an amino acid analog and selecting a variant of the mutant Synechococcus elongatus sp. strain comprises:

plating the mutant Synechococcus elongatus sp. strain on agar containing the amino acid analog and selecting a first variant colony of the mutant Synechococcus elongatus sp. strain that grows in the presence of the amino acid analog;

replating the selected first variant colony and selecting at least a second variant colony of the mutant Synechococcus elongatus sp. strain that grows in the presence of the amino acid analog to obtain segregated variant colonies;

transferring the segregated variant colonies to a liquid medium containing the amino acid analog and selecting for segregated variants that grow in the presence of increasing concentrations of the amino acid analog;

sub-culturing variants from the selected segregated variants in the liquid medium containing the amino acid analog for at least 24-72 hours; and selecting a sub-cultured variant for increased production of the amino acid and less than 5% reduction in biomass accumulation as compared with the wild-type Synechococcus elongatus sp. strain.

Contacting the mutant strain with an amino acid analog and selecting a variant of the mutant Synechococcus elongatus sp. strain can be performed in the presence of light and air supplemented with about 3% v/v CO₂ (such as 3% v/v CO₂). In certain embodiments, contacting the mutant strain with an amino acid and selecting a variant of the mutant strain is performed under ambient CO₂ conditions.

As noted above, in certain embodiments, the selected sub-cultured variant can be subjected to at least one round of mutagenesis. For example, the method can further comprise inducing mutagenesis in the selected variant strain by exposing the selected variant strain to MMS or UV irradiation, or both MMS and UV irradiation to generate a secondary mutant as noted above, and (optionally) contacting the secondary mutant with an amino acid analog and selecting a variant of the secondary mutant having increased production of the amino acid (e.g., as compared to wild-type and/or the first variant colony). In certain embodiments, an additional selection criterion for the secondary mutant can also be less than a 5% reduction in the biomass accumulation as compared to the wild-type Synechococcus elongatus sp. strain.

The method can comprise:

(a) exposing a culture of wild-type Synechococcus elongatus sp. strain to MMS and/or UV irradiation to generate a mutagenized culture (e.g., a mutant Synechococcus elongatus sp. strain);

(b) incubating the mutagenized culture (e.g., a mutant Synechococcus elongatus sp. strain) for at least about 8 hours (such as at least 8 hours) under dark, heated conditions;

(c) plating the mutagenized culture (e.g., a mutant Synechococcus elongatus sp. strain) on agar containing an amino acid analog and selecting a first set of variant colonies that grow in the presence of the amino acid analog;

(d) replating the first set of variant colonies selected in (c) and selecting a second set of variant colonies that grow in the presence of the amino acid analog;

(e) replating the second set of variant colonies selected in (d) and selecting a third set of variant colonies that grow in the presence of the amino acid analog; and, optionally, replating the third set of variant colonies selected in (e) and selecting a fourth set of variant colonies that grow in the presence of the amino acid analog, to obtain completely segregated variant colonies;

(f) transferring the completely segregated variant colonies to a liquid medium containing the amino acid analog and selecting completely segregated variants that grow in the presence of increasing concentrations of the amino acid analog;

(g) sub-culturing variants from (f) in the liquid medium containing the amino acid analog for at least 24-72 hours;

(h) quantifying the amino acid titer (e.g., aromatic amino acid titer (i.e., L-phenylalanine, L-tyrosine, and L-tryptophan)) in a supernatant obtained from a sub-cultured mutant; and

(i) selecting a sub-cultured variant for increased production of the amino acid and less than about a 5% reduction (such as less than a 5% reduction) in biomass accumulation.

The method can further comprise:

(j) obtaining a variant of the sub-cultured variant selected in (i) that outperforms the sub-cultured variant selected in (i) for production of the amino acid.

Cyanobacteria suitable for serving as a starting strain or wild-type/control strain for the mutagenesis and/or selection include members of the genus Synechococcus. The wild-type Synechococcus elongatus can be Synechococcus elongatus sp. PCC11801. Suitable organisms can be obtained from publicly available sources, including by collection from the natural environment. For example, wild-type Synechococcus elongatus sp. PCC11801 can be isolated from Powai Lake (34° C.; pH 7.5) in Mumbai, India (19.1273° N, 72.9048° E) which has a temperature of 34° C. and a pH of 7.5. (see Jaiswal et al. (2018), infra); may be referred to herein as PCC 11801), and has a low doubling time, is resistant to abiotic stressors (e.g., salt and temperature), and has the ability to grow under high light, all of which can be beneficial to produce a strain on a commercial scale. For example, it can grow above 38° C. with a light intensity above 400 μmole photons m⁻².s⁻¹. Optimal growth conditions are 41° C. with a light intensity of 1,000 μmole photons m⁻².s⁻¹ and 0.04% CO₂. It exhibits a doubling time of 2.3 hours under ambient CO₂ conditions. Increasing the level of CO₂ can increase growth under low light conditions but can inhibit growth under high light conditions.

The amino acid can be any amino acid synthesized by a cyanobacteria through a pathway regulated by feedback inhibition where, if the feedback inhibition is removed or reduced, results in an upregulation of the amino acid. The amino acid can be an aromatic amino acid, such as phenylalanine or L-phenylalanine. Where the amino acid is phenylalanine or L-phenylalanine, the amino acid analog can be 3-(2-thienyl)-DL-alanine, a P-fluoro-phenylalanine (p-F-Phe), or any other phenylalanine analog. The amino acid can be tryptophan. Where the amino acid is tryptophan, the amino acid analog can be 5-fluoro-tryptophan or any other tryptophan analog.

In certain embodiments, medium used in the methods hereof for culturing the Synechococcus elongatus sp. variants is a liquid medium, which can comprise components that can promote growth and production of the desired amino acid(s) at commercially practicable scales. The liquid medium can be BG-11 medium. The BG-11 medium can be modified to contain increased concentrations of magnesium sulfate heptahydrate, sodium nitrate, potassium phosphate dibasic, and A5 mineral solution and to include ammonium chloride (“BG-11M medium”). In certain embodiments, the medium used for culturing the Synechococcus elongatus sp. variants to produce one or more amino acid(s) (e.g., L-phenylalanine) comprises a carbon source, an organic or inorganic nitrogen source, and/or any other substances or agents now known or hereinafter discovered that facilitate growth of the Synechococcus elongatus sp. variants.

The incubation in the dark can be at least about 8 hours (such as at least 8 hours), at least about 9 hours (such as at least 9 hours), at least about 10 hours (such as at least 10 hours), at least about 11 hours (such as at least 11 hours), or at least about 12 hours, such as overnight or at least 12 hours. A dark period/cycle can be characterized by low illumination intensities (e.g., illumination at an intensity below a threshold able to drive photosynthesis of the organisms in the culture).

The heated conditions in (b) or in other method embodiments can be about 38° C. to about 40° C. (such as about 38° C. to 40° C. or 38° C. to about 40° C.). Sub-culturing can be at least about 36 hours (such as at least 36 hours), at least about 48 hours (such as at least 48 hours), at least about 60 hours (such as at least 60 hours), or at least about 72 hours (such as at least 72 hours). Quantification of the amino acid titer can involve separation of amino acids by liquid chromatography followed by detection by mass spectrometry (see, for example, et al. (2020), infra), or derivatization followed by gas chromatography and mass spectrometry or liquid chromatography with fluorescent detection.

The organisms or biomass can be harvested by any suitable means, such as centrifugation, flocculation, or filtration, and can be processed immediately or dried for future processing.

Also provided is a variant Synechococcus elongatus sp. obtained by one or more of the above methods, such as when the culture in step (a) of the above method was exposed to UV radiation and/or when mutagenesis was induced in the wild-type Synechococcus elongatus sp. strain by exposing the wild-type Synechococcus elongatus sp. strain to MMS, UV irradiation, or both MMS and UV irradiation. The variant Synechococcus elongatus sp. can be selected from a mutant Synechococcus elongatus sp. PCC 11801 strain (i.e. a mutagenized Synechococcus elongatus sp. PCC 11801). In certain embodiments, the variant can produce at least about 0.5 g/L of an amino acid, such as an aromatic amino acid after three days of culture.

In certain embodiments, the variant Synechococcus elongatus sp. is capable of enhanced production of L-phenylalanine. For example, the mutant Synechococcus elongatus strain can produce at least about 0.5 g/L of L-phenylalanine after three days of culture. In certain embodiments, the variant Synechococcus elongatus sp. can produce at least about 1.2±0.1 g/L of phenylalanine in three days under 3% CO₂. In certain embodiments, the variant Synechococcus elongatus sp. can accumulate up to 3 g/L of L-phenylalanine and 7 gL of biomass after 15 days in culture. If the variant Synechococcus elongatus sp. loses biomass, in certain embodiments, the loss of biomass is less than about 5%, such as less than a 5% reduction.

In certain embodiments, the variant of Synechococcus elongatus sp. comprises a variant of Synechococcus elongatus sp. PCC 11801 identified as [identifier] and deposited at [location] with the deposit reference number [ ]. Such a variant has properties of increased L-phenylalanine production and less than about a 5% reduction in biomass accumulation (such as less than 5%) as compared with the wild-type Synechococcus elongatus sp. strain.

When the method further comprises (j), the variant can produce at least about 0.8 g/L, at least about 1.2 g/L, or up to about 2.0 g/L of an amino acid, such as an aromatic amino acid, e.g., L-phenylalanine, after three days of culture, such as with a biomass production of at least 95% that of the wild-type strain, e.g., when cultured in BG-11M medium. The variant can produce about 3.0 g/L (such as 3.0 g/L) of an amino acid, such as an aromatic amino acid, e.g., L-phenylalanine, after 15 days of culture, such as with a biomass production of at least 95% that of the wild-type strain, e.g., when cultured in BG-11 medium that is periodically replenished.

A method of extending growth of a culture of a variant cyanobacterium and its photoautotrophic production of an amino acid is also provided. The method comprises:

(a) culturing the variant cyanobacterium, which photo-autotrophically produces an amino acid, in BG-11 medium,

(b) supplying the culture with air supplemented with CO₂, and

(c) periodically replenishing the BG-11 medium. The BG-11 medium can be BG-11M medium. The exogenous CO₂ can be at least about 3% v/v (such as at least 3% v/v). The BG-11 medium, such as BG-11M medium, can be replenished every 2-3 days, such as on days 3, 6, 9, 12, and 15. The variant cyanobacterium can be a variant of a mutagenized Synechococcus elongatus sp. strain, such as one that is selected for overproduction of an amino acid, such as an aromatic amino acid, e.g., L-phenylalanine. The variant Synechococcus elongatus can be variant of a mutagenized Synechococcus elongatus sp. PCC11801, such as one that is selected for overproduction of an amino acid, such as an aromatic amino acid, e.g., L-phenylalanine. The culture of a variant S. elongatus sp. PCC11801 can accumulate at least about 7.0 g/L of biomass and at least about 3.0 g/L (such as 3.0 g/L) of an amino acid, such as an aromatic amino acid, e.g., L-phenylalanine, at 15 days in culture. The culture can be viable for longer than 12 days, such as 20 days.

Further provided is a method of photo-autotrophically producing L-phenylalanine. The method comprises culturing a variant of a S. elongatus sp. strain (e.g., which can produce at least about 0.5 g/L, at least about 0.8 g/L, or up to about 2.0 g/L of an amino acid, such as an aromatic amino acid, e.g., L-phenylalanine, after three days of culture or about 3.0 g/L (such as 3.0 g/L) of an amino acid, such as an aromatic amino acid, e.g., L-phenylalanine, after 15 days of culture) under conditions suitable for culturing a Synechococcus elongatus sp. strain to produce L-phenylalanine. The variant of the Synechococcus elongatus sp. strain can be produced by inducing mutagenesis in a wild-type Synechococcus elongatus sp. strain by exposing the wild-type Synechococcus elongatus sp. strain to MMS, UV irradiation, or both MMS and UV irradiation to produce a mutant Synechococcus elongatus sp. strain, and contacting the mutant Synechococcus elongatus sp. strain with an L-phenylalanine analog and selecting a variant of the mutant Synechococcus elongatus sp. strain having increased L-phenylalanine production and less than about a 5% reduction (such as less than a 5% reduction) in biomass accumulation as compared with the wild-type Synechococcus elongatus sp. strain. In certain embodiments, the variant of the mutant Synechococcus elongatus sp. strain is cultured in BG-11 or BG-11M medium in the presence of light and air supplemented with about 3% v/v CO₂ (such as 3% v/v CO₂). The BG-11 or BG-11M medium can be replenished every 2-3 days, such as on days 3, 6, 9, 12, and 15. “Light” or “light conditions” means the presence of illumination at an intensity above a threshold able to drive photosynthesis in the organisms of the culture. In certain embodiments, the presence of light means about 240 μmol-photons m⁻² s⁻¹ light, at least about 400 μmol-photons m⁻² s⁻¹ light, above about 400 μmol-photons m⁻² s⁻¹ light, or at or about 1,000 μmol-photons m⁻² s⁻¹ light.

The amino acid, e.g., L-amino acid, such as an aromatic amino acid, e.g., L-phenylalanine, can be collected from the medium at any point during culture or at the end of the culture period (e.g., when the culture has stopped growing and/or stopped producing the amino acid). No special method is required. For example, the amino acid, e.g., L-amino acid, such as an aromatic amino acid, e.g., L-phenylalanine, can be concentrated and crystallized after removing cells from the medium or by ion-exchange chromatography. Additionally or alternative, the organisms, biomass, and/or amino acid can be harvested by any suitable means, such as centrifugation, flocculation, or filtration, and can be processed immediately or dried for future processing (as appropriate and/or desired).

The amino acid, e.g., L-amino acid, such as an aromatic amino acid, e.g., L-phenylalanine, can be purified by a combination of conventionally known ion-exchange resin methods (see, e.g., Nagai et al., Separation Science and Technology 39(16): 3691-3710 (2004)), membrane separation methods (see, e.g., Japanese Patent Laid-Open Nos. 9-164323 and 9-173792), crystallization methods (see, e.g., Int'l Pat. App. Pub. Nos. WO 2008/078448 and WO 2008/078646), and other methods.

### EXAMPLES

The following examples serve to illustrate the present disclosure. The examples are not intended to limit the scope of the claimed invention.

### Example 1

### Development of Phenylalanine-Overproducing Mutants

A modified BG-11 recipe (BG-11M) was developed and used for culture as described in Tables 1 and 2. BG-11M was developed by increasing the amounts of magnesium sulfate heptahydrate (increased from 75 mg/L to 95 mg/L), sodium nitrate (increased from 1.5 g/L to 2.5 g/L), potassium phosphate dibasic (increased from 40 mg/L to 80 mg/L), and A5 mineral solution (increased from 1 ml/L to 2 ml/L) in BG-11 and adding ammonium chloride (10 mg/L). For making plates, 1.5% Difco Bacto agar (Becton, Dickinson and Co., Franklin Lakes, N.J.) was added. Sterilization was achieved by autoclaving on wet cycle for 20 minutes at 121° C.

TABLE 1
BG-11M solution recipe for 1 L

| Component | Amount | |
|---|---|---|
| Sodium Nitrate (NaNO₃) | 2.50 | g |
| Magnesium sulfate heptahydrate (MgSO₄•7H₂O) | 95 | mg |
| Calcium chloride dihydrate (CaCl₂•2H₂O) | 36 | mg |
| Sodium carbonate (Na₂CO₃) | 20 | mg |
| Citric acid (C₆H₃O₇) | 6 | mg |
| Potassium phosphate dibasic (K₂HPO₄) | 80 | mg |
| Ferric ammonium citrate (C₁₂H₂₂FeN₃O₄) | 6 | mg |
| EDTA (C₁₀H₁₆N₂O₈) | 1 | mg |
| Ammonium chloride (NH₄Cl) | 10 | mg |
| A5 mineral solution (added after autoclave) | 2 | mL |

TABLE 2
A5 mineral solution recipe for 1 L

| Component | Amount | |
|---|---|---|
| Ultrapure water | 1 | L |
| H₃BO₃ | 2.86 | g |
| MnCl₂•4H₂O | 1.81 | g |
| ZnSO₄•7H₂O | 0.222 | g |
| NaMoO₄•2H₂O | 0.39 | g |
| CuSO₄•3H₂O | 0.079 | g |
| Co(NO₃)₂•6H₂O | 49.4 | mg |

The wild-type strain of Synechococcus elongatus sp. PCC 11801 (Jaiswal et al. (2018), infra) was obtained from Dr. Wangikar, Indian Institute of Technology, Bombay, India. The wild-type and all mutant strains were routinely cultured (50 mL volume) in the modified BG-11 media (hereafter BG-11M) in 250 mL Erlenmeyer flasks at 38° C. with 240 μmol-photons m⁻² s⁻¹ light and a supply of air or 3% (vol/vol) CO₂-enriched air in an incubator shaker (Infors HT Minitron, Infors AG, Bottmingen, Switzerland) set at 200 rpm. The growth of the cultures was determined by measuring either the optical density at 730 nm (OD₇₃₀; using a Beckman DU Series 500 spectrophotometer) or the dry cell weight. The relationship between the dry cell weight and the OD₇₃₀ was 0.401 g (dry weight) cells liter⁻¹ OD₇₃₀⁻¹.

Random mutagenesis was performed by modifying a previously described protocol (Deshpande et al. (2020), infra) with methylmethanesulfonate (MMS) and ultra-violet (UV) irradiation as the mutagens. Cells were prepared for random mutagenesis by pelleting exponentially growing cultures (OD ˜0.6-1) of PCC 11801 and resuspending in 1 mL of fresh BG-11M media.

For MMS mutagenesis, 1% (v/v) MMS (Sigma-Aldrich, St. Louis, Mo.) was added for 90 seconds, and the reaction was quenched by the addition of 5% (w/w) sodium thiosulfate solution to yield a ˜85-95% kill rate. For UV mutagenesis, the culture was subjected to 90 seconds of UV light to yield a kill rate of ˜90%.

The mutagenized cultures were incubated at 38° C. overnight in the dark before selection. To select for overproducers, the phenylalanine (Phe) analog 3-(2-thienyl)-dl-alanine (Sigma-Aldrich, St. Louis, Mo.) was applied to BG-11M agar plates in concentrations ranging from 0.5-2 mg/ml. The wild-type and mutagenized cultures were diluted and spread on BG-11M (to test kill rate) and BG-11M analog-containing plates, and were incubated at 38° C. until colonies appeared. Colonies (i.e. variant Synechococcus elongatus sp. strains) that successfully grew on analog selection plates were picked (e.g., selected) and re-streaked on selective plates for at least three rounds to enable complete segregation of mutations.

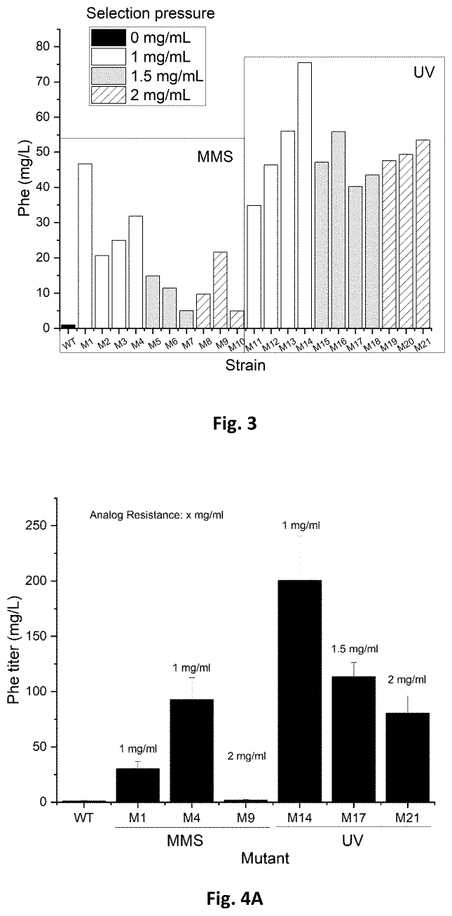

21 selected variants were transferred to liquid media with the same analog concentration used on the plates. The variants were selected to include both UV and MMS mutants that were resistant to 1 mg/ml, 1.5 mg/ml and 2 mg/ml analog (FIG. 3). After subculture and inoculation at the same starting concentration, aromatic amino acid (AA) titers in the supernatants of three days-old cultures were quantified as previously described (Deshpande et al. (2020), infra).

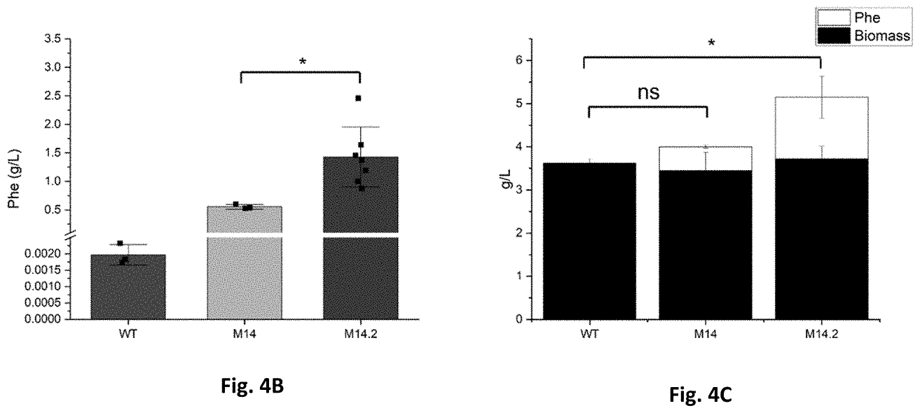

The highest amino acid-producing mutant strains (MMS mutants: M1, M4, and M9 and UV mutants: M14, M17, and M21) were characterized (in triplicate) for biomass accumulation (e.g., to ensure stable phenotype after removal of selection pressure) and Phe production (FIG. 4A). M14 was identified as the best strain from the first round of random mutagenesis. Thus, this variant Synechococcus elongatus sp. strain was selected for another round of mutagenesis using both MMS and UV as mutagens. UV and 1% MMS (v/v) exposures were tested at 60 sec, 90 sec and 120 sec, coupled with selection on plates containing 1.5-3 mg/ml of the Phe analog 3-(2-thienyl)-dl-alanine. After screening several resistant colonies as described previously, a colony was identified as outperforming the parent strain M14 for Phe production. This colony was a result of 60 sec UV exposure and selection on 1.5 mg/mL analog. This mutant variant, denoted as M14.2, accumulated Phe ranging from 0.8-2 g/L in 3 days, a significant improvement over M14 (roughly 2-fold Phe in the media as compared to M14) (FIG. 4C). The biomass accumulation was not affected (FIG. 4B). Further rounds of streaking led to a stable phenotype with Phe production 1.24±0.13 g/L. The mutant variant was tested and found to be resistant up to 3 mg/L of analog on selective plates.

### Example 2

### Prolongment of Culture Viability and Phenylalanine Production

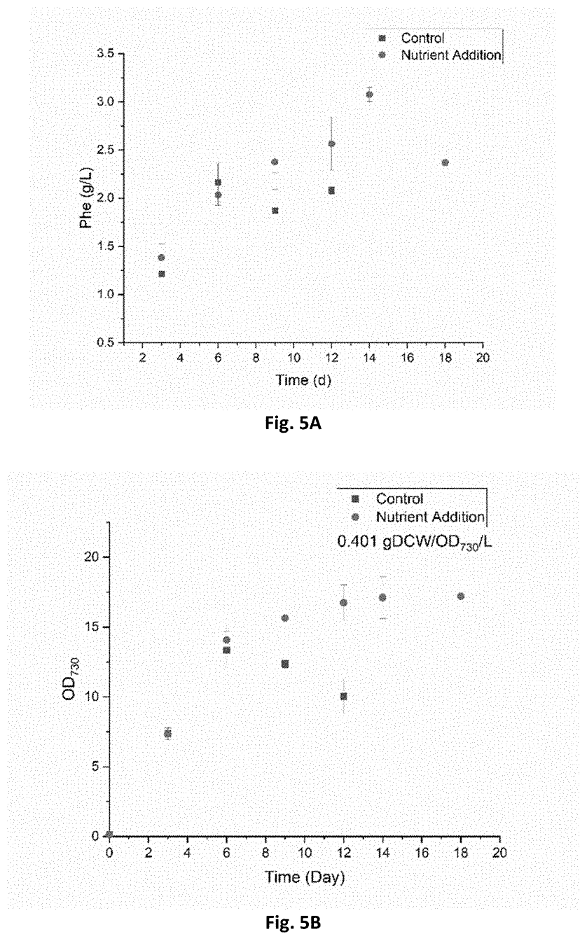

A method was developed to prolong the culture viability and Phe production phase under the above culture conditions with 3% CO₂ v/v and an initial culture density of OD₇₃₀ 0.2. The method utilized the addition of 1.25 mL of 40× concentrated BG-11M solution to 50 mL cultures to replenish complete BG-11M nutrients every three days (days 3, 6, 9, 12, and 15). This resulted in enhanced accumulation of biomass (˜7 g/L) and Phe (˜3 g/L) at 15 days in culture, when compared to control where nutrients were not replenished (FIGS. 5A and 5B). This strategy enabled culture viability for 20 days as opposed to 12 days.

### Example 3

### Effect of Light on Carbon Fixation and Phenylalanine Overproduction

Scaling up and testing cyanobacteria cultures outdoors requires robust performance under stressors such as high light and day-night cycles. While the aforementioned segregation and Phe characterization studies were performed under continuous illumination of 240 μmol-photons m⁻² s⁻¹, the performance of the present mutant variant strains was also tested under a 12 h:12 h light dark cycle and separately at a higher light intensity (i.e., closer to expected outdoor culture conditions). Accordingly, three light conditions were tested.

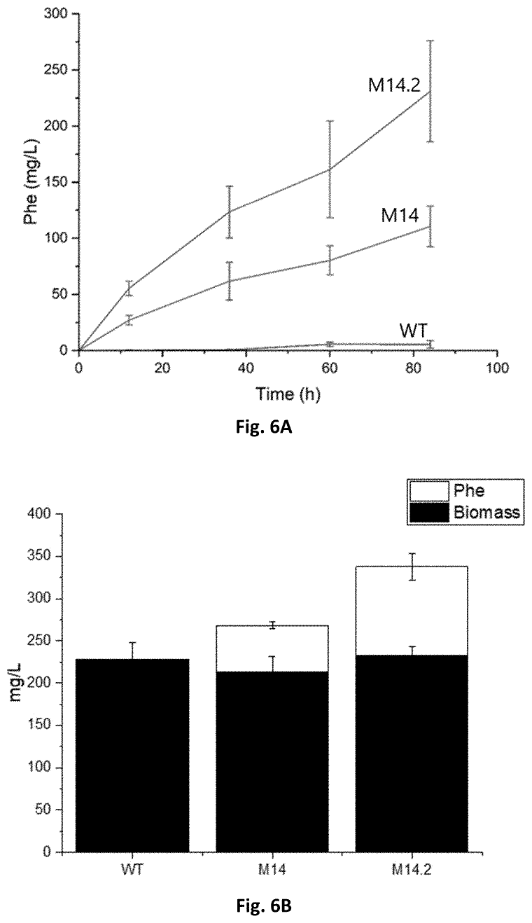

Both M14 and M14.2 were adapted to at least one subculture under light dark cycle before being tested for Phe production (FIG. 6A) and biomass accumulation (FIG. 6B). The Phe productivity normalized to time under illumination was comparable when cultured in a 12 h:12 h light dark cycle and under continuous illumination from both M14 and M14.2. The biomass accumulation for both the mutant variant strains was comparable to wild-type, supporting that it is unlikely that under dark conditions Phe is uptaken to be used as either a nitrogen or carbon source.

To determine strain performance under high light conditions, 1045±45 240 μmol m⁻²s⁻¹ continuous illumination was used. Due to higher availability and greater penetration of light at higher cell densities, M14.2 accumulated 287±28 mg/L Phe in 3 days, with the resultant productivity 95±9 mg/L/day Phe being the highest reported in cyanobacteria under ambient CO₂. At 240 μE, Phe production was achieved by increased carbon fixation by mutant variants (e.g., by greater photosynthetic efficiency) and did not affect biomass.

### Example 4

### Effect of Dissolved Concentration of CO₂ on Phenylalanine Overproduction

Enhancing dissolved concentration of CO₂ has been shown to increase not only the growth rate but also the final culture density of wild-type Synechococcus elongatus sp. PCC 11801 (Jaiswal et al. (2018), infra). Previously aromatic amino acid production was found to be enhanced under the supply of CO₂ enriched air (Deshpande et al. (2020), infra; Brey et al. (2020), infra). To improve final product and biomass titer and productivities, the effect of a 3% CO₂ supply was tested.

FIG. 4A shows the Phe titers of the mutant variant strains M14 and M14.2 as compared to wild-type Synechococcus elongatus sp. PCC 11801. To ensure stability of the most productive strain M14.2, the experiment was repeated three times and M14.2 had a consistent phenotype with a Phe titer 1.24±0.13 g/L in 3 days. FIG. 4B shows the total accumulation of two major sink products, biomass and Phe. Both M14 and M14.2 showed higher carbon fixation productivities compared to wild-type by nearly 14% and 28%, respectively, although only M14.2 was statistically significant at p<0.05.

There was no significant difference in biomass accumulation or composition of the wild-type (data not shown) and mutant variants. Phe production was instead enhanced by fixing carbon at a higher rate, which supports that under 3% CO₂ supply, Synechococcus elongatus sp. PCC 11801 is sink limited and the introduction of a strong sink for Phe relieves this limitation and can result in improved carbon fixation. This phenomenon has also been observed in other cyanobacteria, where strains engineered for production of sucrose, 2-phenylethanol, or 2,3-butanediol exhibited increased carbon fixation.

### Example 5

### Nutrient Supplementation Using BG-11M

Improving biomass accumulation can be vital to increasing the titer of growth associated products such as Phe. Recently, the biomass accumulation of Synechococcus sp. PCC 6803 and Synechococcus sp. PCC11901 were improved by alleviating nutrient limitations (van Alphen et al. (2018), infra; Włodarczyk et al. (2020), infra). The contents of BG-11M were tested for optimization by further optimizing levels of nitrates, phosphates, sulfates as well as nutrient replete 5× BG-11M. None of these resulted in improved performance compared to BG-11M media (data not shown).

The addition of complete BG-11M nutrients every 3 days was then tested, using a concentrated solution to minimize volume changes. This strategy resulted in an increase in the biomass and Phe accumulation to nearly 7 g DCW/L and 3 g/L, respectively, in 15 days under 240 μmol-photons m⁻² s⁻¹ light and 3% CO₂ as shown in FIG. 5A and FIG. 5B. This result outlines a strategy that can be easily implemented at higher scales to reduce the number of batches.

### Example 6

### Comparison of Phenylalanine Productivity in Engineered Cyanobacteria

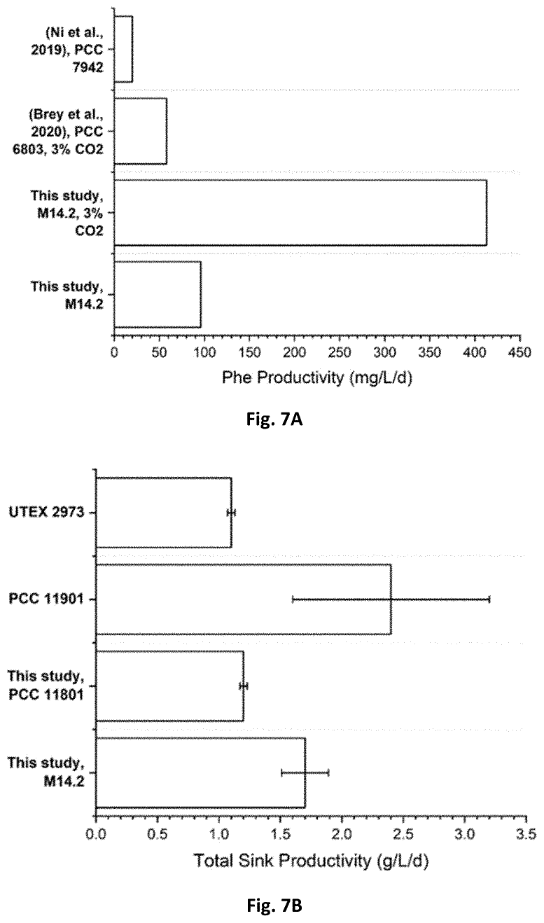

The methods hereof (e.g., using random mutagenesis on Synechococcus elongatus sp. PCC 11801) developed Phe overproducing strains that accumulated up to 3 g/L Phe, the highest reported titer in cyanobacteria to date. FIGS. 7A and 7B compare the Phe productivity in cyanobacteria engineered for production of Phe and its derivatives. As compared to other approaches, mutant variant strain M14.2, exhibited a higher productivity under both ambient and 3% CO₂ conditions, with a maximum productivity about 400 mg/L/d Phe under a three-day cultivation cycle. Further, total carbon sink productivity was one of the highest reported in cyanobacteria and comparable to other fast growing cyanobacteria strains under high CO₂ as shown in FIG. 7B.

The comparison with heterotrophs is not a direct one. Heterotrophic Phe producers such as E. coli must utilize sugars such as glucose and suffer from low yields on glucose, typically lower than 50% of the maximum theoretical yield of 0.55 g/g. (Liu et al. (2018), infra). Accordingly, when the two-step process of sugar production by crops and subsequent conversion to Phe by heterotrophs is compared with the single-step cyanobacterial process using space-time yield as previously described, heterotrophs fall short. The current best Phe producing bacterial strain can accumulate roughly 72 g/L Phe in the media with a yield of 0.26 mol/mol in glucose (Liu et al. (2018), infra). Assuming the average yield for sugarcane over the last 10 years (70.63 tfw/ha/annum) and a sugar content of 15% (FAO, 2020), the space-time yield for the two-step process is nearly 7 kgPhe/ha/d. In the case of cyanobacterial photoautotrophic Phe production, commercial photobioreactors of scale 500,000 L/ha can reach the same cell density as reported in FIG. 4B with productivity 21% as compared to shake flasks. This results in a space-time yield of nearly 22 kgPhe/ha/d, which is about three-fold more efficient than the current heterotrophic process. Table 3 provides a summary of the space-time yields of each approach.

TABLE 3
Comparison of Space-Time Yields

| | Space-time yield |
|---|---|
| Organism | (kgPhe/ha/d) |
| M14.2 | 22* |
| E. coli | 7.5** |
| Plants | 0.23*** |

*Assuming scale up in outdoor photobioreactors (500,00 I/ha), productivity 0.2 × shake flask due to D/N and efficiency losses
**Highest reported titer 72 g/L, 0.26 mol glucose yield (Liu et al. (2018), infra) data for sugarcane productivity and sugar yield.
***Phe derived from soy protein fraction, av yield/ha/annum

### Certain Definitions

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

The term “about” as used herein can allow for a degree of variability in a value or range, for example, within 10%, within 5%, or within 1% of a stated value or of a stated limit of a range.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

The terms and expressions, which have been employed, are used as terms of description and not of limitation. In this regard, where certain terms are defined under “Certain Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Certain Definitions,” are used in the “Detailed Description,” such use is solely for ease of reference and is not intended to limit any disclosure made in one section to that section only; rather, any disclosure made under one subheading is intended to constitute a disclosure under each and every other subheading.

In the description hereof, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary, but remain applicable in view of the data provided herein.

Wherever feasible and convenient, like reference numerals are used in the figures and the description to refer to the same or like parts or steps. The drawings are in a simplified form and not to precise scale. It is understood that the disclosure is presented in this manner merely for explanatory purposes and the principles and embodiments described herein may be applied to assembly components that have configurations other than as specifically described herein. Indeed, it is expressly contemplated that the components of the methods, mutants and variants of the present disclosure may be tailored in furtherance of the desired application thereof.

In describing representative embodiments, a method and/or process may have been presented as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

Further, all publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls. Full citations of the references cited herein are provided below.

### REFERENCES

1. Bongaerts et al., Metabolic Engineering for Microbial Production of Aromatic Amino Acids and Derived Compounds. Metabolic Engineering 3(4): 289-300 (2001). doi: 10.1006/mben.2001.0196.
2. Brandenburg et al., Trans-4-hydroxy-L-proline production by the cyanobacterium Synechocystis sp. PCC 6803. Metabolic Engineering Communications 12, e00155 (2021). doi: 10.1016/j.mec.2020.e00155.
3. Brey et al., Metabolic engineering of Synechocystis sp. PCC 6803 for the production of aromatic amino acids and derived phenylpropanoids. Metabolic Engineering 57: 129-139 (2020). doi: 10.1016/j.ymben.2019.11.002.
4. Deshpande et al., Combining Random Mutagenesis and Metabolic Engineering for Enhanced Tryptophan Production in Synechocystis sp. Strain PCC 6803. Applied and Environmental Microbiology 86(9) (2020). doi: 10.1128/aem.02816-19.
5. FAO, World Food and Agriculture—Statistical Yearbook 2020, FAO (2020).
6. Hall and Jensen, Enzymological Basis for Growth Inhibition by 1-Phenylalanine in the Cyanobacterium Synechocystis sp. 29108. Journal of Bacteriology 144(3): 1034-1042 (1980). doi: 10.1128/jb.144.3.1034-1042.1980.
7. Jaiswal et al., Cyanobacteria as cell factories: the roles of host and pathway engineering and translational research. Current opinion in biotechnology 73: 314-322 (2021). Advance online publication. https://doi.org/10.1016/j.copbio.2021.09.010.
8. Jaiswal et al., Genome Features and Biochemical Characteristics of a Robust, Fast Growing and Naturally Transformable Cyanobacterium Synechococcus elongatus PCC 11801 Isolated from India. Sci Rep 8: 16632 (2018).
9. Liu et al., Metabolic engineering for the production of 1-phenylalanine in Escherichia coli. 3 Biotech 9(3): 85 (2019). https://doi.org/10.1007/s13205-019-1619-6.
10. Liu et al., Genetic engineering of Escherichia coli to improve L-phenylalanine production. BMC Biotechnology 18(1) (2018). doi: 10.1186/s12896-018-0418-1.
11. Ni et al., Remodeling of the Photosynthetic Chain Promotes Direct CO 2 Conversion into Valuable Aromatic Compounds. Angewandte Chemie International Edition 57(49): 15990-15994 (2018). doi: 10.1002/anie.201808402.
12. Oliver and Atsumi, A carbon sink pathway increases carbon productivity in cyanobacteria. Metabolic Eng'g 29: 106-112 (2015). doi: 10.1016/j.ymben.2015.03.006.
13. Riccardi et al., Amino acid biosynthesis and its regulation in cyanobacteria. Plant Science 64(2): 135-151 (1989). doi: 10.1016/0168-9452(89)90018-6.
14. Rippka et al., Generic Assignments, Strain Histories and Properties of Pure Cultures of Cyanobacteria. Microbiology 111(1): 1-61 (1979). doi: 10.1099/00221287-111-1-1.
15. Santos-Merino et al., Improved photosynthetic capacity and photosystem I oxidation via heterologous metabolism engineering in cyanobacteria. Proceedings Of The National Academy Of Sciences 118(11): e2021523118 (2021). doi: 10.1073/pnas.2021523118.
16. Schipper et al., Outdoor scale-up of Leptolyngbya sp. Effect of light intensity and inoculum volume on photoinhibition and -oxidation. Biotechnology And Bioengineering 118(6): 2368-2379 (2021). doi: 10.1002/bit.27750.
17. Singh et al., Emerging platforms for co-utilization of one-carbon substrates by photosynthetic organisms. Current Opinion in Biotechnology 53: 201-208 (2018). doi: 10.1016/j .copbio.2018.02.002.
18. van Alphen et al., Increasing the photoautotrophic growth rate of Synechocystis sp. PCC 6803 by identifying the limitations of its cultivation. Biotechnol. J. 13: 1700764 (2018).
19. Włodarczyk et al., Newly discovered Synechococcus sp. PCC 11901 is a robust cyanobacterial strain for high biomass production. Communications Biology 3(1) (2020). doi: 10.1038/s42003-020-0910-8.
20. Zhang et al., Adaptive Laboratory Evolution of Microalgae: A Review of the Regulation of Growth, Stress Resistance, Metabolic Processes, and Biodegradation of Pollutants. Frontiers In Microbiology 12 (2021). doi: 10.3389/fmicb.2021.737248.

What is claimed is:

1. A method of generating a variant cyanobacterium for photoautotrophic production of an amino acid, which method comprising:
inducing mutagenesis in a wild-type Synechococcus elongatus sp. strain by exposing the wild-type Synechococcus elongatus sp. strain to methylmethanesulfonate (MMS), ultraviolet (UV) irradiation, or both MIMS and UV irradiation to generate a mutant Synechococcus elongatus sp. strain; and
contacting the mutant Synechococcus elongatus sp. strain with an amino acid analog and selecting a variant of the mutant Synechococcus elongatus sp. strain having increased production of the amino acid and less than about a 5% reduction in biomass accumulation as compared with the wild-type Synechococcus elongatus sp. strain.

2. The method of claim 1, wherein the wild-type Synechococcus elongatus is Synechococcus elongatus sp. PCC11801.

3. The method of claim 1, wherein the amino acid is phenylalanine.

4. The method of claim 1, wherein the amino acid is L-phenylalanine.

5. The method of claim 4, wherein the amino acid analog is 3-(2-thienyl)-DL-alanine.

6. The method of claim 1, wherein contacting the mutant strain with an amino acid analog and selecting a variant of the mutant Synechococcus elongatus sp. strain further comprises:
plating the mutant Synechococcus elongatus sp. strain on agar containing the amino acid analog and selecting a first variant colony of the mutant Synechococcus elongatus sp. strain that grows in the presence of the amino acid analog;
replating the selected first variant colony and selecting at least a second variant colony of the mutant Synechococcus elongatus sp. strain that grows in the presence of the amino acid analog to obtain segregated variant colonies;
transferring the segregated variant colonies to a liquid medium containing the amino acid analog and selecting for segregated variants that grow in the presence of increasing concentrations of the amino acid analog;
sub-culturing variants from the selected segregated variants in the liquid medium containing the amino acid analog for at least about 24-72 hours; and
selecting a sub-cultured variant for increased production of the amino acid and less than 5% reduction in biomass accumulation as compared with the wild-type Synechococcus elongatus sp. strain.

7. The method of claim 6, wherein the liquid medium is BG-11 medium.

8. The method of claim 7, wherein the BG-11 medium is modified to contain increased concentrations of magnesium sulfate heptahydrate, sodium nitrate, potassium phosphate dibasic, and A5 mineral solution and to include ammonium chloride (BG-11M medium).

9. The method of claim 6, further comprising
inducing mutagenesis in the selected sub-cultured variant by exposing the selected sub-cultured variant to MMS, UV irradiation, or both MMS and UV irradiation to generate a sub-cultured mutant; and
contacting the selected sub-cultured mutant with an amino acid analog and selecting a variant of the selected sub-cultured mutant having increased production of the amino acid and less than about a 5% reduction in biomass accumulation as compared with the wild-type Synechococcus elongatus sp. strain.

10. The method of claim 9, wherein the selected sub-cultured mutant is exposed to MMS and UV irradiation for a period of about 60 seconds to about 120 seconds.

11. The method of claim 1, further comprising incubating the mutant Synechococcus elongatus sp. strain for at least about 8 hours under dark, heated conditions.

12. The method of claim 11, wherein heated conditions are about 38° C. to about 40° C.

13. The method of claim 1, wherein contacting the mutant strain with an amino acid analog is performed in the presence of light and air supplemented with about 3% v/v carbon dioxide (CO₂).

14. A variant of Synechococcus elongatus sp. obtained by the method of claim 1.

15. The variant of claim 14, which is a variant of a mutagenized Synechococcus elongatus sp. PCC11801.

16. The variant of claim 14, which can produce at least about 0.5 g/L of L-phenylalanine after three days of culture.

17. The variant of claim 16, which can accumulate up to 3 g/L of L-phenylalanine and 7 g/L of biomass after 15 days of culture.

18. A method of photo-autotrophically producing L-phenylalanine, which method comprises:
culturing a variant of a Synechococcus elongatus sp. strain under conditions suitable for culturing a Synechococcus elongatus sp. strain to produce L-phenylalanine, the variant of Synechococcus elongatus sp. strain produced by:
inducing mutagenesis in a wild-type Synechococcus elongatus sp. strain by exposing the wild-type Synechococcus elongatus sp. strain to methylmethanesulfonate (MMS), ultraviolet (UV) irradiation, or both MMS and UV irradiation ultraviolet to produce a mutant Synechococcus elongatus sp. strain, and
contacting the mutant Synechococcus elongatus sp. strain with an L-phenylalanine analog and selecting a variant of the mutant Synechococcus elongatus sp. strain having increased L-phenylalanine production and less than about a 5% reduction in biomass accumulation as compared with the wild-type Synechococcus elongatus sp. strain.

19. The method of claim 18, wherein the variant of the mutant Synechococcus elongatus sp. strain is cultured in BG-11 or BG-11M medium in the presence of light and air supplemented with about 3% v/v CO₂.

20. The method of claim 18, further comprising collecting L-phenylalanine from a biomass of the cultured variant or a culture medium thereof.
